(19) United States
(12) Patent Application Publication
(10) Pub. No.: US 2006/0142706 A1
(43) Pub. Date: Jun. 29, 2006

(54) Ocular device for current controlled variable delivery of active principles

(76) Inventors: Pierre Roy, Paris (FR); Alain Kleinsinger, Neuilly-Sur-Seine (FR)

Correspondence Address: BLAKELY SOKOLOFF TAYLOR & ZAFMAN/PDC, 12400 WILSHIRE BOULEVARD, SEVENTH FLOOR, LOS ANGELES, CA, 90025, US

(21) Appl. No.: 10/559,633

(22) PCT Filed: Jun. 3, 2004
(86) PCT No.: PCT/IB04/02052

§ 371 Date: Dec. 1, 2005

(30) Foreign Application Priority Data
Jun. 3, 2003 (FR) 03/06677

(51) Int. Cl. A61M 35/00

(52) U.S. Cl. 604/296

(57) ABSTRACT

Device for delivering active principles to the eye, comprising a reservoir able to contain the active principles, and means for releasing the active principles contained in the reservoir towards the vicinity of a site intended to receive the active principles, characterized in that the device also comprises means for distributing the active principles which can be driven by iontophoresis or electroporation.

The invention relates to devices for the continuous and/or discontinuous delivery of any substance presented as having curative or preventive properties with regard to human diseases of the eye, in particular intra- and extra-ocular devices.

Ocular devices for delivering active principles, capable of delivering said active principles in the long term (from several days to several weeks, or even several months) in a continuous and/or discontinuous manner are provided in two forms:
Inserts are reservoirs of active principles, placed at the surface of the eyeball non-invasively, such as contact lenses, preferably in the conjunctival sac. They allow continuous or programmed delivery of active principles. Many systems have been developed, either in the form of a lens or of a ring, or in the form of a small annular or tubular reservoir placed in the conjunctival sac, usually in the inferior fornix. A disadvantage of the inserts is limited passage of the active principles into the posterior segment of the eyeball, limiting their use to the treatment of pathologies affecting the anterior segment of said eyeball (inflammation, conjunctivitis). In addition, there is a relatively high risk of the insert being ejected, accidentally or otherwise.
Intra-ocular implants for the programmed release of active principles are inserted surgically into the vitreous body of the eyeball. Such implants may or may not be biodegradable/bioerodable. This type of device is able to move around freely in said vitreous body and there is a risk of it affecting the retina by locally increasing the concentration of active principles, which could reach a toxic level. It is, however, possible to suture the implant, but this requires a relatively large incision (approximately 5 mm). Another drawback of non-biodegradable implants is having to replace the implant regularly in order to provide delivery of active principles in the very long term.

The disadvantage of these devices, whether they are inserts or implants, is that it is not possible to interrupt or to accelerate the treatment according to the progression of the pathology to be treated. In fact, these devices exhibit a profile for delivery of active principles which is fixed over time. This profile is defined either at the time of manufacture, or before the device is placed in the eye. A change in delivery profile requires replacement of the device. This becomes very prejudicial for the patient in the case of intravitreous implants since this requires a surgical procedure.

In the case of a mechanism for controlled delivery by means of a diffusion—controlled delivery mechanism, the active principles are enclosed in a reservoir, for example, the walls of which have a certain porosity, or alternatively within “delayed-action” layers which dissolve in the environment, agents maintaining the active principles or containing the active principles dissolving in water, or even being biodegradable in nature. These diffusion—control mechanisms, referred to as passive, allow continuous, stable, increasing or decreasing delivery profiles, or even profiles for delivery in several sequences. However, these profiles are always preprogrammed. In addition, they are difficult to set up since they are affected by the parameters of the environment or else of the change in dimensions of the substrate (swelling, dissolution), which modifies the specific surface area for diffusion for example.

In the case of a mechanism for delivery by means of a chemical effect, the control of delivery consists in binding the active principles to one or more substrates, the substrate(s) biodegrading, or the chemical bond rupturing in situ. It is then possible to link the delivery mechanism to temperature and/or to pH. This makes it possible to release the active principle in the presence of certain surrounding conditions. These mechanisms can be triggered on demand, by the introduction of external energy such as heat or light, heating or electrolysis making it possible to modify the pH. This makes it possible to insert the implant into a tumour, for example, and to trigger the release.

In the case of mechanical delivery mechanisms, the latter mainly make use of the osmotic principle consisting in enclosing the active principle in a reservoir with a polymer which swells under the effect of water (or which generates a gas) and which ejects the active principle out of said reservoir. Other mechanisms of this type-use springs, gas or various agents which expand according to various initiating mechanisms and make it possible to generate a force to propel the active principle generally contained within an envelope. One way to initiate or to accelerate the delivery of an active principle contained in such an implant is to use a directly applied electric current, or radiation, for heating a polymer, which changes from the crystalline state to the molten state, and thus releaseing the active principle trapped in a crystalline matrix. The radiation used can be light such as that emitted by a laser beam. Ultrasound can also be used to modify the flow of active principle through the porous wall of a reservoir.

However, all these control devices require complex and delicate equipment for insertion and use.

An aim of the invention is to provide a device for delivering active principles continuously and/or discontinuously in the eye, which allows a variable delivery profile and which is simple to use.

For this, the invention provides a device for delivering active principles to the eye; comprising a reservoir able to contain the active principles, and means for releasing the active principles contained in the reservoir around the vicinity of a site intended to receive the active principles, characterized in that the device also comprises means for distributing the active principles which can be controlled by iontophoresis or electroporation.

Advantageously, but optionally, the device has at least one of the following characteristics:
the distribution means are a microporous wall,
the distribution means contain valves, the opening of which is controlled by iontophoresis or electroporation,
the distribution means comprise an electrically sensitive polymer or gel capable of modifying the volume of the reservoir containing the active principles under the action of iontophoresis or electroporation,
the distribution means contain at least one polymer gel containing the active principles, which can be eroded under the effect of iontophoresis or electroporation,
the distribution means also contain electrodes which extend by protruding out of the device in such a way as to allow anchoring of the device when it is put in place,
the device comprises a second reservoir of active principles,
the device is an insert which can be placed on the surface of an eyeball,
the insert can be placed in a conjunctival sac,
the device is an implant which can be placed inside the eyeball.

Other characteristics and advantages of the invention will emerge from the description hereinafter of a preferred embodiment and also of variants. In the attached drawings:

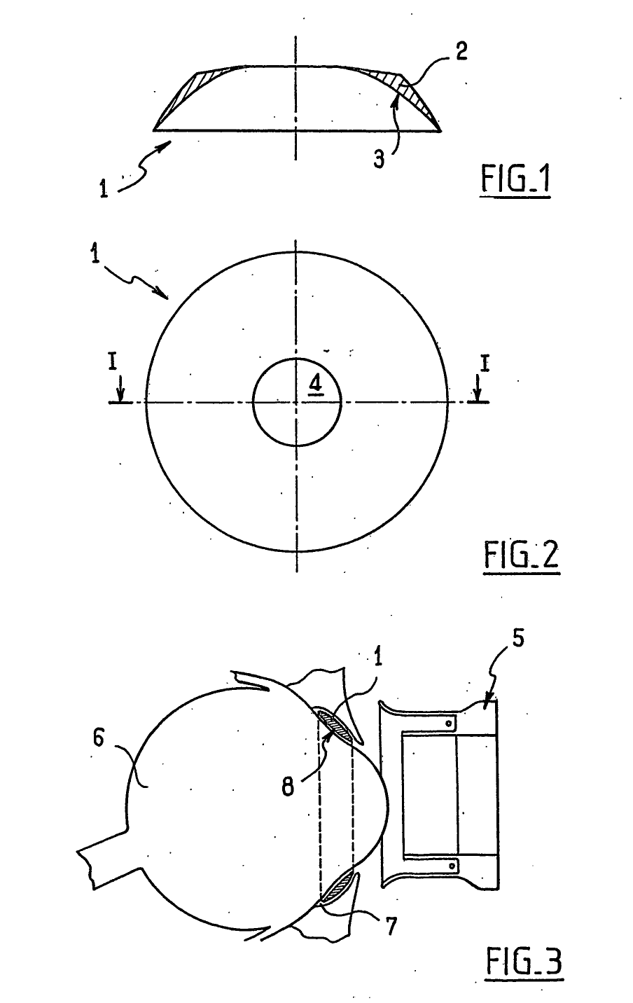

FIG. 1 and FIG. 2 are, respectively, a sectional view and a view from above of an insert according to the invention,

FIG. 3 is a diagrammatic view of the use of the insert of FIGS. 1 and 2,

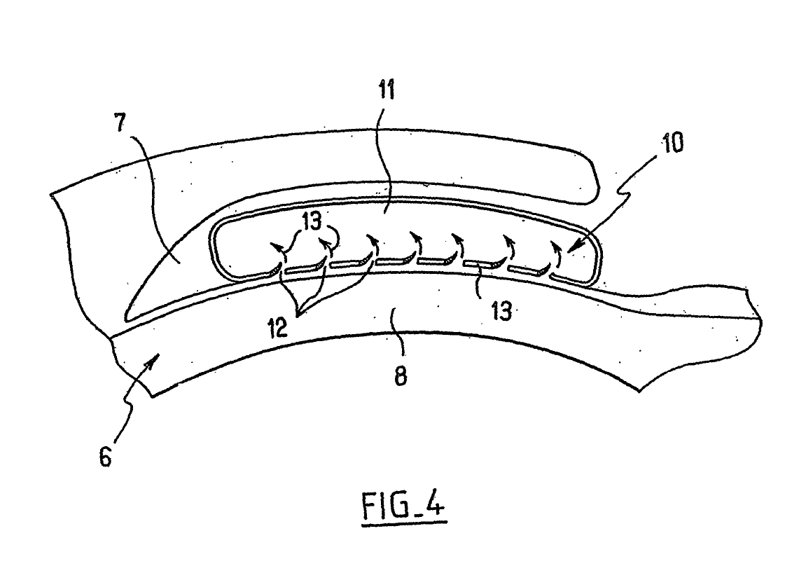

FIG. 4 is a sectional view of an insert according to a second embodiment implanted in the conjunctival sac of a patient,

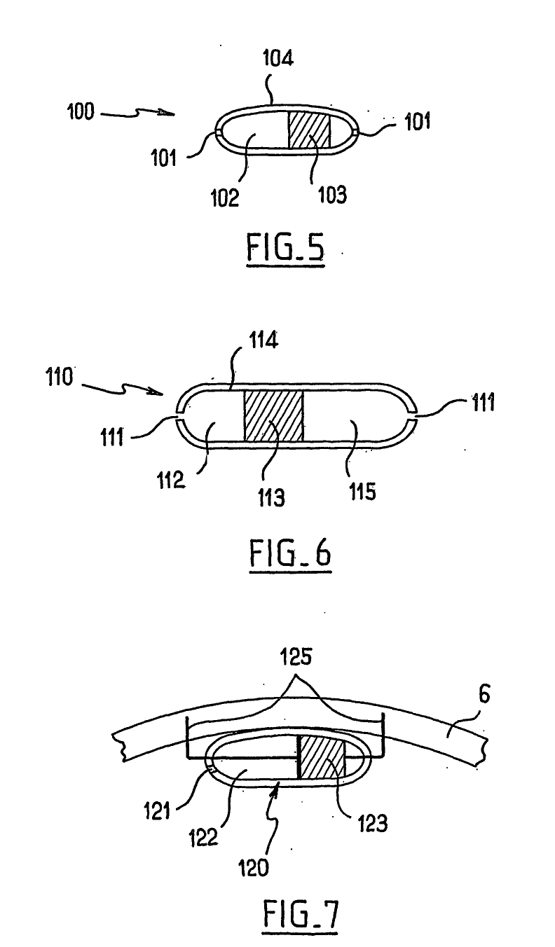

FIG. 5 is a sectional view of an embodiment of an implant according to the invention,

FIG. 6 is a sectional view of a variant of implementation of an implant according to the invention,

FIG. 7 is a sectional view of a second variant of implementation of an implant according to the invention,

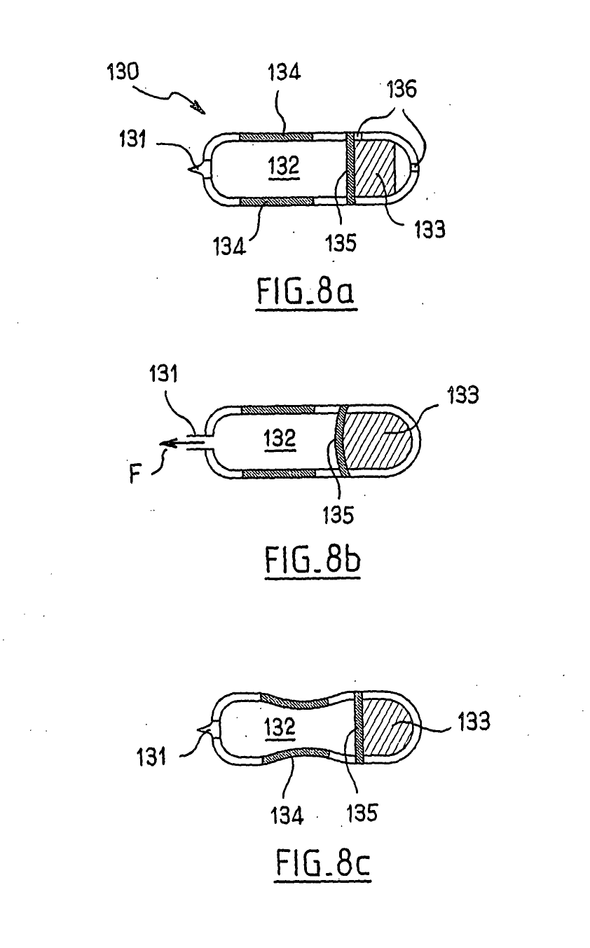

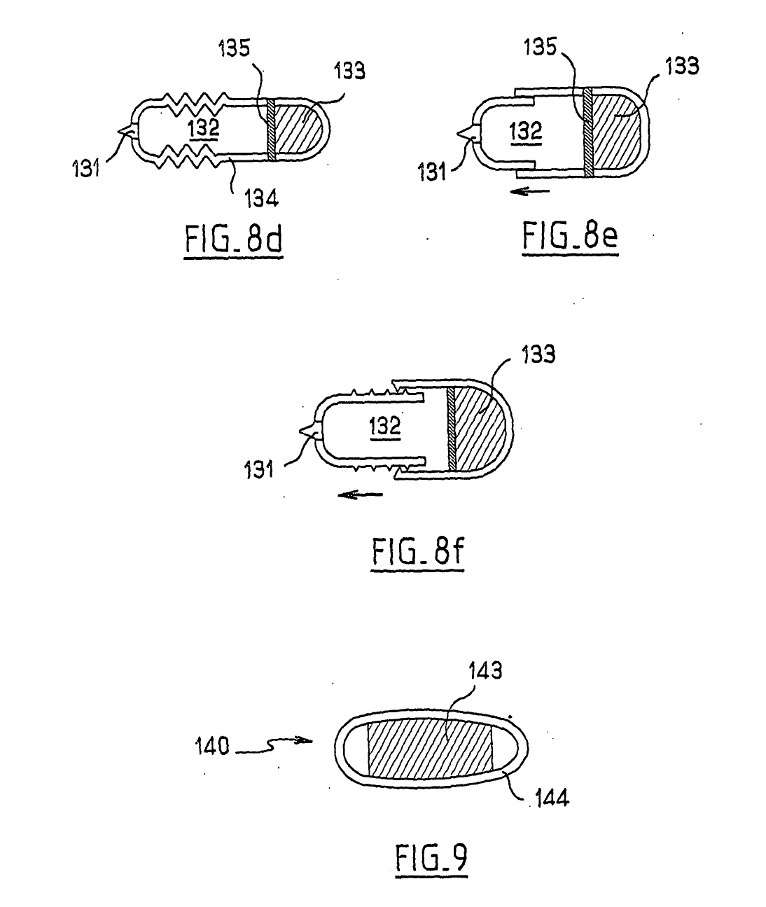

FIGS. 8a to 8c are sectional views of a third variant of implementation of an implant according to the invention,

FIGS. 8d, 8e and 8f illustrate three other variants of implementation based on similar principles, and

FIG. 9 is a sectional view of a fourth variant of implementation of an implant according to the invention.

We will describe a device according to the invention, provided in the form of an insert, with reference to FIGS. 1 to 4.

We will describe a first embodiment of an insert according to the invention with reference to FIGS. 1 to 3. The insert 1 is, in this case, provided in the form of an annular lens comprising a central orifice 4 and a reservoir able to contain one or more active principles intended to be delivered to the eyeball. In addition, the insert 1 in the form of a lens has an internal face 3 which is convex in shape and substantially complementary to the shape of the outer surface of the anterior sclera of the eye, namely the conjunctiva with which it is intended to cooperate so as to able to deliver the active principles contained in the reservoir 2.

With reference to FIG. 3, the insert 1 described above is applied against an eyeball 6 at the level of the sclera 8 of said eyeball 6. Thus, the reservoir 2 of the insert comes to be placed in the conjunctival sac 7 of the eyeball 6. This form of insert is preferable since, in this way, it covers the greatest possible surface area of the sclera 8 while at the same time being maintained in the conjunctival sac 7. The insert 1 releases the active principle(s) contained in its reservoir 2 in a continuous manner since the surface 3 of the insert 1 is microporous and allows the active principles to pass through. However, a large portion of the active principles thus administered is washed away by tears, while the other portion remains in the anterior segment of the eye after having crossed the sclera 8. The intermittent application of an applicator for ocular iontophoresis 5 makes it possible to increase the scleral permeability for several hours and thus to allow a greater portion of the active principles to cross the scleral wall 8, thus avoiding these said active principles being washed away by tears. An applicator for ocular iontophoresis is known per se. More information on this type of applicator can be found in document FR 2 773 320 or document FR 2 830 766. Thus, by adjusting the scleral permeability by means of an iontophoretic current, it is possible to modify the delivery profile of the insert 1. In addition, applicators for ocular iontophoresis are simple and easy to use: the patient can himself or herself apply the prescribed treatment and thus modify the delivery profile of his or her insert 1.

With reference to FIG. 4, we will describe a second embodiment of an insert according to the invention. The insert 10, which has any shape, but which can have the same shape as the insert 1 described above, has a reservoir 11 able to contain one or more active principles intended to be delivered through the scleral wall 8 of an eyeball 6. As above, the insert 10 is placed in the conjunctival sac 7 of the eyeball 6. The reservoir 11 of the insert 10 is delimited internally by a surface 13 which can be in direct contact with the scleral wall 8. Preferably, the wall 13 of the reservoir 11 is microporous so as to continually deliver the active principle(s) contained in said reservoir under simple osmotic pressure. Furthermore, the microporous wall 13 of the reservoir 11 contains specific pores 12 which are resting in the closed position and which are capable of opening according to a movement 13 under the action of an iontophoretic current. The specific pores 12 act as valves. These valves can be produced according to one of the following ways:
coating of the polymers with a conductive film or a conductive ink, or even with a film deposited by vacuum sputtering or bombardment, etc., or alternatively
addition of a conductive charge, in the form of metal or carbon-based powder or fibre, to the polymer forming the valve, or alternatively
production of the valve by means of intrinsically conductive polymers such as polyacetylene, polypyrrole, polythiophene or polyaniline and, in general, any electrically conductive material (metallic materials could in particular also be suitable).

These polymers can be formed and arranged as valves inside the reservoir as is illustrated in FIG. 4. Under the action of the iontophoresis, the valves 12 lift up and release the active principle(s) in much greater amount at the surface of the eyeball at the level of the scleral wall 8.

In one variant of implementation, the valves can be formed with electrically sensitive polymers which are capable of changing shape under the action of an iontophoretic current.

In a third embodiment of an insert according to the invention (not shown), the insert consists of a hydrogel containing the active principles in an encapsulated manner. The hydrogel can be produced in two ways:
it is a polymeric matrix containing the active principle(s) which become(s) released under the action of the iontophoretic current, or
it is a gel made of polymer that is erodable under the action of an iontophoretic current.

In the three embodiments described above, it should be noted that the insert is prepared in a preferably annular form (complete or partial ring), preserving the cornea which is the functional surface of the eye and located opposite the orifice 4. In addition, this annular form enables the insert to press against the sclera 8 around said cornea, which sclera is slightly vascularized and substantially thinner in this area than the rest of the wall of the eyeball 6.

Furthermore, the applicator for ocular iontophoresis is placed opposite the sclera, either directly on the insert or else on the eyelids, which are then closed and thus preserve the cornea.

Other variants of implementation are of course also possible for the insert.

In particular, it can also be advantageously envisaged for the insert to be an annular-shaped insert divided up into several sectors and each containing a different active principle, for example an anti-inflammatory and an antibiotic for treating both endophthalmitis and the inflammatory reaction generally subsequent to the eye infection.

With reference to FIGS. 5 to 9, we will describe a device according to the invention provided in the form of an implant.

With reference to FIG. 5, we will describe a first embodiment of such an implant. The implant 100 contains a reservoir 102 able to contain one or more active principles. The reservoir has walls 104 which are substantially microporous so as to allow water (making up approximately 98% of the vitreous body in which the implant is intended to be placed) to pass through and also the ions that are in this water. In a variant of implementation, the wall 104 can contain calibrated through-orifices 101 which allow the reservoir 102 to be in communication with the outside of the implant 100. Furthermore, the reservoir 102 contains, within itself, an electrically sensitive polymer or hydrogel 103. The particularity of such an electrically sensitive polymer or such an electrically sensitive hydrogel is to have the property of changing shape or of expanding under the action of an iontophoretic current. Such polymers or hydrogels are thus referred to as electrorheological gels, or alternatively piezoelectric materials. This type of gel can be produced by adding fine polarizable particles to a fluid having a lower dielectric constant, for example silicone oil. Under the effect of an iontophoretic current, the polarizable particles align and, as a result, change the rheological properties of the fluid comprising them. This effect is called the Winslow effect.

The implant 100 thus prepared is inserted into the vitreous body of an eyeball and releases a dose of active principles in a linear and continuous manner under simple osmotic pressure. Under the effect of the application of an iontophoretic current, by means of an iontophoretic device as mentioned above, the electrically sensitive polymer 103 expands and expels a bolus of active principles for a selected period of time. Then, once the iontophoretic current has been turned off, the polymer returns to the initial state and the implant 100 continues to release a dose in a continuous and linear manner under the effect of osmotic pressure, as before the application of an iontophoretic current. It should be noted that the presence of the orifices 101 makes it possible to ensure a good electrical contact between the aqueous humour and the gel so as to improve the reaction of the polymer 103 under the effect of an iontophoretic current.

In addition, the tubular shape of the device 100 makes it possible to control and to increase the dose of active principles released, even for very small variations in volume of the electrically sensitive polymer or gel 103. However, it is possible to use other reservoir shapes such as rings, discs or spheres, for example.

With reference to FIG. 6, we will describe a second embodiment of an implant according to the invention.

The implant 110 contains two reservoirs 112 and 115 delimited by walls 114 which may be microporous. The orifices 110 allow direct communication between the environment in which the implant 110 is implanted and the content of the reservoirs 112 and 115, respectively. An electrically sensitive polymer or gel 113 makes it possible to simultaneously distribute the content of the two reservoirs 112 and 115. As above, the electrically sensitive polymer changes shape under the effect of an iontophoretic current.

With reference to FIG. 7, we will describe a third embodiment of an implant according to the invention. The implant 120 has a reservoir 122 similar to the reservoir 102 above, and also an electrically sensitive gel or polymer 123 similar in its role to the polymer 103 above. An orifice 121 makes it possible to bring the aqueous humour of the vitreous body into contact with the content of the reservoir 122. Furthermore, the implant 120 has electrodes 125 which come into contact on both sides with the electrically sensitive polymer or gel 123 and extend by protruding out of the implant 120. The ends which are outside the implant 120 make it possible to anchor the device in the wall of an eyeball 6 when it is implanted. Thus, the electrodes 125 have a double role of maintaining the implant 120 in place and making it possible to increase the iontophoretic current density when such a current is applied, the result of which is to increase the expansion of the electrically sensitive polymer 123 and its effect as regards the bolus of active principles which is ejected out of the implant 120.

With reference to FIGS. 8a to 8f, we will now describe a fourth embodiment of an implant according to the invention. The implant 130 has a reservoir 132 delimited by a wall comprising semi-flexible parts 134.

In addition, the reservoir 132 has a non-return valve 131 for establishing communication between the outside and the inside of the reservoir 132. Finally, the reservoir 132 is separated from an electrically sensitive polymer or gel 133 by a flexible membrane 135. On the polymer 133 side, the wall of the device surrounding said polymer has orifices 136. Under the effect of an iontophoretic current illustrated in FIG. 8b, the electrically sensitive polymer 133 changes shape by expanding while pushing the flexible membrane 135, thus reducing the volume of the reservoir 132, the content of which, thus increasing under pressure, opens the valve 131 and releases a bolus of active principles in the direction of the arrow F towards the outside. Once the iontophoretic current has been turned off, the electrically sensitive polymer 133 returns to its initial shape, as does the flexible membrane 135. Under the decrease in pressure which is then created in the reservoir 132, the non-return valve 131 closes and the semi-flexible walls 134 themselves also change shape, in order to compensate for said reduced pressure. The implant 130 then has the shape illustrated in FIG. 8c.

As a further variant, and as illustrated in FIGS. 8d, 8e and 8f, the semi-flexible parts 134 can be replaced with parts in the form of bellows (FIG. 8d) or with a structure where the implant consists of two parts, one of which slides into the other (FIG. 8e), where appropriate with means of a notch or ratchet type (FIG. 8f).

In a fifth embodiment of an implant according to the invention, illustrated in FIG. 9, the implant 140 contains a gel 143 surrounded by a microporous wall 144. The gel 143 is a gel or a matrix containing the active principle(s) and is in addition sensitive to the iontophoretic current. This makes it possible to release the principle(s) according to the following two principles:
either a polymeric matrix containing the active principles, which are then released under the action of the iontophoretic currents, is used, or
a gel made of polymer which has the particularity of being erodable under the action of the iontophoretic current is used.

In a variant of implementation of this embodiment of an implant according to the invention, the gel 143 can be replaced with a plurality of gels having the same properties under the effect of an iontophoretic current, each of the gels comprising a different active principle. Similarly, in another variant of implementation, the implant 140 can have electrodes similar to the electrodes 125 in the embodiment described above, making it possible to anchor the implant 140 in the scleral wall and to increase the iontophoretic density and therefore the effect of this current on the gel 143.

Of course, many modifications may be introduced into the invention without however departing from the scope thereof.

1. Device for delivering active principles to the eye, comprising a reservoir able to contain the active principles, and means for releasing the active principles contained in the reservoir towards the vicinity of a site intended to receive the active principles, characterized in that the device also comprises means for distributing the active principles which can be driven by iontophoresis or electroporation.

2. Device according to claim 1, characterized in that the distribution means are a microporous wall.

3. Device according to claim 1 or 2, characterized in that the distribution means contain valves, the opening of which is driven by iontophoresis or electroporation.

4. Device according to one of the preceding claims, characterized in that the distribution means comprise an electrically sensitive polymer or gel capable of modifying the volume of the reservoir containing the active principles under the action of iontophoresis or electroporation.

5. Device according to one of claims 1 to 3, characterized in that the distribution means contain at least one polymer gel containing the active principles, which can be eroded under the effect of iontophoresis or electroporation.

6. Device according to claim 4 or 5, characterized in that the distribution means also contain electrodes which extend by protruding out of the device in such a way as to allow anchoring of the device when it is put in place.

7. Device according to claim 6, characterized in that the device comprises a second reservoir of active principles.

8. Device according to one of claims 1 to 7, characterized in that the device is an insert which can be placed on the surface of an eyeball.

9. Device according to claim 8, characterized in that the insert can be placed in a conjunctival sac.

10. Device according to one of claims 1 to 7, characterized in that the device is an implant which can be placed inside the eyeball.
